(19) United States
(12) Patent Application Publication
(10) Pub. No.: US 2024/0068039 A1
(43) Pub. Date: Feb. 29, 2024

(54) MULTI-VECTOR DETECTION OF VARIANT SEQUENCES

(71) Applicant: Saga Diagnostics AB, Lund (SE)

(72) Inventors: Miguel Alcaide, Lund (SE); Anthony Miles George, Lund (SE); Lao Hayamizu Saal, Lund (SE)

(21) Appl. No.: 18/240,416

(22) Filed: Aug. 31, 2023

Related U.S. Application Data
(60) Provisional application No. 63/402,507, filed on Aug. 31, 2022.

(51) Int. Cl. C12Q 1/6886; G01N 33/58

(52) U.S. Cl. CPC C12Q 1/6886; G01N 33/58; C12Q 2600/118; C12Q 2600/156

(57) ABSTRACT

The present invention provides a method for detecting variant nucleic acid sequences, which are often found in low-abundance, including structural sequence variants and mutations using dPCR and patterns in one or more two-color plots, comprising a multi-vector representation, indicative of a particular variant sequence.

### TECHNICAL FIELD

The present invention relates to the field of methods for detection of low abundance target nucleic acids.

### BACKGROUND

Many diseases and conditions, particularly cancers, are associated with variant DNA sequences. Many of these variant sequences are somatic variants, which occur in the non-germline cells. While many germline variants are associated with a pathogenic condition, they do not necessarily contribute to disease progression or symptomology. Nevertheless, evidence has emerged showing that many diseases and conditions, whether in an individual or across a population, exhibit patterns of variant sequences. Consequently, detecting variant sequences or patterns of variant sequences may provide insights, including information about disease progression/regression, treatment efficacy, minimal residual disease (MRD), origins of a tumor, specific defects in DNA repair, certain environmental exposures, optimal treatments for a condition, and may be a foundation for personalized medicine.

However, while detecting variant sequences has clear utility, in many circumstances, especially in the context of somatic variants, these variants are only present in small quantities. Thus, exceptionally sensitive and specific methods for mutation detection are necessary, in particular for low-input samples such as circulating cell-free DNA (cfDNA) and analysis of single cells. Conventional methods can suffer from a constellation of issues, including a high requirement of input sample DNA quantity, high per-sample cost, complex and laborious workflows, insufficient sensitivity and/or specificity, and inability to detect low-abundance mutated sequences within a high background of normal wild-type sequence (so-called mutant allele fraction; MAF). One conventional solution is digital PCR in PCR reactions are partitioned into many smaller individual reactions so that each reaction partition contains zero to only a very few target sequence molecules. This results in a potential increase in sensitivity. However, conventional dPCR often fails to detect the presence of low abundance target nucleic acids in a biological sample due to the comparatively weak signals provided by low abundance nucleic acids, such as variant sequences caused by cancers, and due to false-positives stemming from sources such as noise or DNA damage. The failure to detect low abundance sequences is more pronounced when using traditional dPCR to simultaneously detect multiple variant sequences in a single assay.

### SUMMARY

The present invention provides methods and systems for the rapid identification of variant sequences or patterns of variant sequences, despite their low abundance a sample. According to the invention, variant sequences are detected using a digital polymerase chain reaction (dPCR) that produces identifiable fingerprints that enable variant detection using many, several, only a few, or even a single, detectable label to identify multiple variants. Methods of the invention utilize multi-dimensional plots to inform a multi-vector representation of variants in a sample. The plots are produced using dPCR that uses a first set of probes, each of which has a first detectable label and is specific for a different variant sequence; and an additional set or sets of probes having a second or third (or more) detectable label. The amplicons are produced in partitions from which the labels are detected. Any suitable detectable label may be used. For example, in some embodiments, the detectable labels are optical labels, such as from fluorescently-labelled hydrolysis probes, a multi-color plot may be produced from detected optical signals for identification of variant sequences based on a deviation from an expected wild-type cluster plot. According to this method, the invention is amenable to greater than two sets of probes to elucidate one or more plots that can be compared to expected wild-type cluster plots. Preferred embodiments will be presented in the form of two-color plots for ease of demonstration, but the skilled artisan understands that additional multi-color plots may be used in accordance with the invention.

In certain aspects, the invention provides methods for detecting variant nucleic acids. According to the invention, a sample suspected of containing nucleic acid targets is partitioned and nucleic acid in the partitions are amplified in the presence of a set of variant-specific probes, wherein each variant-specific probes includes a detectable label and wherein the entire set of probes includes a number of distinct detectable labels that may be lower than the number of variants to be detected. Variants are detected from amplicons in the partitions. A plot of points representing the optical signals is generated such that the presence of variants in the sample is identified based on a corresponding cluster of points in the plot.

Preferred methods may include mapping the detected signals onto a space defined by the number of distinct detectable labels. Preferred methods include assigning a vector to each cluster of points, wherein each vector uniquely and specifically identifies one of the variants in the sample. In some embodiments, at least one of the probes detects all of the variants in the sample, and at least a second of the probes is specific to fewer than all of the variants and is present at a different concentration than the first probe. The second probe is used to discriminate among the variants by causing points in the plot to form distinct clusters. Two or more of the different variant sequences may occur at positions on the target nucleic acid that will be amplified by one primer pair in the amplifying step.

The presence of one or more variant sequences may indicate a disease state, such as cancer. In certain embodiments, the presence of one or more variant sequences is indicative of minimal residual disease. In one embodiment, methods of the invention include obtaining an estimate of the relative abundances of the variants prior to the partitioning the sample and designing the variant-specific probes based on the estimate. The presence or absence of one or more of the variant sequences indicates a progression or regression of the disease state. The variants may include tumor mutations determined by sequencing tumor nucleic acid from a tumor sample. In some embodiments, digital PCR further uses probes specific for a wild-type sequence.

Some embodiments include assigning the variants to tranches and, for at least one tranche, providing at least one probe that detects all variants in the tranche and at least one probe that discriminates between variants in the tranche. Tranches may be determined based on genomic position or information about probable relative abundance of the variants in the sample. For example, each tranche may be defined as a set of variants that can be amplified by one primer pair.

In certain embodiments, the detectable labels are optical labels. In some embodiments, optical labels for use in the invention are selected from FAM, HEX, SUN, VIC, TAMRA, ATTO550, Cy5, ROX, ATTO700, Cy5.5, Yakima Yellow, ABY, or JUN (e.g., on fluorescent hydrolysis probes such as TAQMAN probes). The sample may be cell-free DNA (cfDNA).

Prior to aliquoting, the invention may include identifying a first pair of first and second variants among the variants that form overlapping clusters on a 2D dPCR plot and designing a probe set that includes: (i) detection probes for both variants of the pair, wherein the detection probes both have a detection optical label of a first color; and (ii) at least one discrimination probe having a discrimination optical label of a second colors. Amplification may be performed with the detection probes and the discrimination probe present at different concentrations. The detection probes may further include any number of additional probes and labels such as, for example, a third probe specific for a third variant, not of the pair, the third probe having a detectable label, such as an optical label of the first color. The plot may include first, second, and third clusters from the respective first, second, and third variants, and wherein vectors from the origin of the plot through centroids of the clusters are non-orthogonal. Preferably the first, second, and third variants are located at positions on the target nucleic acid so as to all be amplified by one primer pair during the amplifying step.

In certain embodiments, the probes include: a first probe specific for a first variant and having a first optical label; a second probe specific for a second variant and having the first optical label; a third probe specific for a third variant and having the first optical label; a WT probe specific for a wildtype sequence and having a second optical label; and a discrimination probe specific for the first variant and having a third optical label. A first two-color plot from the first and second optical labels is generated, and the presence of at least the first and second variants from first two-color plot is identified. The method may further include generating a second two-color plot from the first and third optical labels, and discriminating the first variant sequence from at least the second variant based on a deviation of the second two-color plot from an expected dPCR two-color plot.

In other aspects, the present application provides method for detecting variant nucleic acids. An exemplary method includes partitioning a sample comprising target nucleic acids into reaction partitions and performing digital PCR on in the reaction partitions. The dPCR reactions may be performed using a first set of probes that are each specific for a different variant sequence and have a first optical label, and one or more probes specific for a different sequence and having a one or more different optical labels. Using a dPCR assay optical signals are detected from the probes in each partition. The signals from the, for example, wildtype and variant sequence probes are used to generate two-color plots. Subsequently, the presence of one or more variant sequences may be identified based on a deviation in the plotted signal from an expected wildtype cluster plot. This deviation may be a recognized pattern in the multi-vector representation, characteristic of a particular variant sequence, whether in a subject or historically associated with a particular disease or condition.

In certain methods, the first set of probes includes a probe having the first optical label and specificity for a first variant sequence and a probe having a third optical label and specificity for the first variant sequence. Such methods may further include generating a two-color plot from the first optical label and third optical label to identify the presence of the first variant sequence based on a shift in the deviation. In such cases, the third optical label may be used to help discriminate one or more of the variant sequences.

Optionally, in some embodiments, probe labels may be assigned to variants based on information about the variant frequencies, e.g., population frequencies, disease frequencies, allele frequencies, or mutant allele fraction (MAF). For example, variants can be assigned to tranches or categories based on their frequencies or MAF. This allows, for example, very rare (low frequency) variants to be assigned certain labels. For example, two rare variants and a relatively abundant variant that are close to each other (e.g., polymorphisms at the same base or within a few, e.g., 20, bases of each other) may all three be given the same optical label on their respective probes, and then may be given additional discrimination probes with different optical label(s). In some of the above embodiments, the probe that comprises the third optical label is specific for a variant sequence with a higher frequency/MAF relative to the different variant sequences of the first set of probes. Such methods may further include the use of a wildtype-sequence-specific probe without a label; a second set of probes that are each specific for a different variant sequence and have a third optical label; a third set of probes that are each specific for a different variant sequence and have a fourth optical label; and/or a fourth set of probes that are each specific for a different variant sequence and have a fifth optical label.

Using methods of the invention, it is possible to detect two or more different variant sequences that occur at the same genomic position of a target nucleic acid. In certain aspects, one or more variant sequences detected by methods and systems of the invention indicates a diseased state, such as cancer. Alternatively or additionally, the presence of one or more variant sequences indicates minimal residual disease (MRD).

In some methods of the invention, the relative frequencies or concentration (number of variant copies per unit of volume) of one or more variants appearing in a target nucleic acid are known for a particular disease or condition. These frequencies or MAF or concentration for the variants may be measured at a first time for a subject and that information may be used in designing probes and labels for a later assay (e.g., dPCR). Then, at a later second time point, those designed probes and labels may be used to assay a sample from the subject for those variants. Alternatively or additionally, the variant sequences are recurrent variant sequences of the diseased state amongst a population, and may have a known, historical frequency. In either situation, identifying the presence or absence of one or more of the variant sequences may indicate a progression or regression of the diseased state—for example, by detecting MRD or a change in frequencies in response to a treatment.

In certain methods of the invention, the step of performing digital PCR further includes using a second set of probes that are each specific for a different variant sequence and have a third optical label, wherein the first and second set of probes are specific for distinct variant sequences. In this way, the multiplex capability of inventive methods is expanded. In such circumstances, each set of probes is specific for a tranche of different variant sequences having similar relative frequencies during the diseased state. The present Inventors discovered that, while not necessary, dividing variant sequences into tranches of similarly-frequent variants helps provide more robust detection of the sequences using a single label for each tranche.

In preferred aspects, labels used in the methods of the invention include on or more labels selected from, but not limited to, FAM, HEX, VIC, SUN, Yakima Yellow, CY5, CY5.5, ROX, ABY, TAMRA, and ATTO550.

Methods of the invention may be used when the sample comprising target nucleic acids includes highly fragmented target nucleic acids. For example, methods of the invention may be used when a sample comprises cell-free DNA (cfDNA) and/or DNA from a formalin-fixed, paraffin-embedded (FFPE) tissue sample. Advantageously, the methods of the invention may be used with probes bind to amplicons of less than 65 bp in length during the digital PCR step. It is noted, that this size is well under the typical size for cfDNA, allowing the methods to be used for these highly fragment, yet critically important nucleic acids.

### BRIEF DESCRIPTION OF THE DRAWINGS

FIG. 1 shows an exemplary method of the invention.

FIG. 2 summarizes the probes, variants, and labels of the exemplary method of FIG. 1.

FIG. 3 provides two-color plots from dPCR reactions to identify variant sequences.

FIG. 4 provides two-color plots from dPCR reactions to identify variant sequences.

FIG. 5 provides composite two-color plots from dPCR reactions to identify variants.

FIG. 6 provides schematic fingerprints or patterns for dPCR results.

FIG. 7 provides composite two-color plots from dPCR reactions to identify variants.

FIG. 8 provides two-color plots from dPCR reactions to identify variant sequences.

FIG. 9 provides composite two-color plots from dPCR reactions to identify variants.

FIG. 10 provides two-color plots from dPCR reactions to identify variant sequences.

FIG. 11 provides exemplary workflows according to the invention.

FIG. 12 provides example probe concentrations for example designs to identify variants.

### DETAILED DESCRIPTION

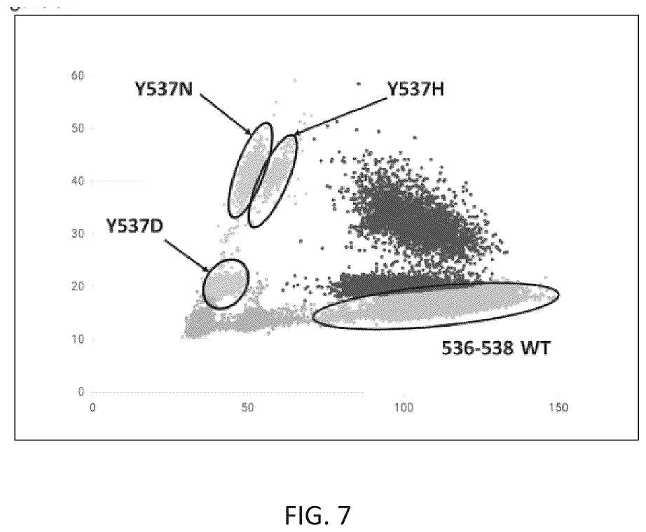

The present invention provides methods for detecting low-abundance nucleic acids, including structural variants and mutants. In preferred embodiments, a sample is subject to multiplex digital PCR (dPCR) with a set of probes that uniquely identifies each variant in a sample. Multiplex dPCR according to the invention reveals the presence and quantities of a number of variants in a sample, even when the number of variants is larger than a number of distinct optical labels used on the probes. In one illustrative example, shown in FIG. 7, two colors can distinctly identify the presence of three different variants. Continuing with FIG. 7 as an introductory example, three variants are specifically identified with two colors because each variant appears as a cluster on a 2D plot of fluorescent intensity for each partition imaged. Briefly, amplicons carrying the variants of interest are diluted and divided into partitions (such as droplets or microwells). Each partitions contains fluorescently-labeled hydrolysis probes specific to each variant, and primers to amplify the amplicons. The partitions are thermocycled and fluorescent intensity for the two colors is measured. Each partition is drawn as a single one of the points on a 2d plot, e.g., FIG. 7. Those points appear in well-defined clusters that are specific to the variants. A number of points in each cluster provides a measure of a quantity of the variants in the sample. In one example, the number of points is multiplied by a dilution factor to yield a quantity (e.g., moles) of amplicons carrying the in the sample. Giving the quantity over sample volume gives the molar concentration. Because those variant concentrations are given for the numerous variants, the dPCR assay gives a measure of the relative abundances of those variants.

Noting that each cluster on the 2D plot may be represented by a vector in 2D space, methods of the invention are extendable to higher dimensionality. Probe sets may be used that have three, four or more distinct colors to quantify arbitrarily higher numbers of variants (e.g., 7, or 10, or 15, or 25 or more) in a single dPCR assay. Fluorescence from each probe is measured from each partition, and each partition is represented as a point in multi-dimensional space where the number of dimension may be the number of different colored probes (although some embodiments may use one or more channels as reference channels or for some other housekeeping purpose that is not used to form a multidimensional space). Each partition is represented by a point in multi-dimensional space. Any given variant present in numerous of the partitions will show up as a cluster of points in that space. Each cluster may be represented by a vector, which may be determined by a regression through the cluster, a principal component analysis, a centroid of the cluster, or similar. Thus, each variant defines a unique vector in the multidimensional space. Because the vectors are unique (and in particular have unique orientations with respect to the coordinate system), they can be distinguished even if the number of vectors is greater than the number of dimensions. For example, four colors of probes (e.g., FAM, HEX, Cy5, ROX) can be used to plot partitions as points in 4-dimensional space and some higher number of variants (e.g., 5, or 8, or 15 . . . ) can each be uniquely identified by its respective cluster and/or vector in the space. A number of points in the cluster can be used to determine an abundance of that variant in the sample. Thus the relative abundance of the variants is determined.

The multiplex dPCR assay may be used for a variety of purposes. In one example, the assay is used to detect the presence of a disease after treatment. In this example (and there may be numerous others), a tumor biopsy from a subject is subject to genomic sequencing to identify somatic mutations specific to that tumor. The sequencing may reveal tumor specific mutations and may also reveal mutations of different abundance or MAF. Later, after the subject undergoes a treatment, a sample from the subject is assayed by multiplex dPCR according to methods of the disclosure. The dPCR reveals the presence or absence of those specific tumor mutations and their relative abundances. The assay uses a relatively inexpensive and minimally invasive blood draw sometimes referred to as a liquid biopsy. The dPCR from the liquid biopsy reveals remission or residual disease.

Certain optional embodiments of the disclosure use a variant enrichment sample preparation to preferentially increase the number of copies of the structural variants or mutants, which may be present in low concentration in the sample, prior to the dPCR. The sample preparation step, which functions to incrementally amplify variants of interest, followed by dPCR, results in reduction of false-negative results from the sample. As a result, methods of the current invention enhance the sensitivity for sample detection, especially in detecting the presence of low-abundance targets in the biological sample.

Methods of the invention are especially useful for detection of low-abundance target nucleic acids in a sample. Preferably, a target nucleic acid is any nucleic acid sequence the presence of which is desirable to detect, including one or more variant sequences of interest. The target nucleic acid or variant may for example be a nucleic acid sequence associated with a clinical condition. In particular, target nucleic acid sequences are variant sequences, which are often associated with early tumorigenesis (a truncal variant).

The present invention includes methods for detecting variant nucleic acids. An exemplary method includes partitioning a sample from a subject comprising target nucleic acids into reaction partitions and performing digital PCR in the reaction partitions. The dPCR reactions may be performed using sets of probes. Preferably, the probes are nucleic acid probes with optically detectable labels (e.g., hydrolysis or Taqman probes). Probes within each set have the same optical label, but the optical labels may differ between probes of different sets. Further, in each set of probes, the probes are specific for, and thus hybridize to, different target nucleic acid variant sequences. Additionally, for a reference, a probe for a wildtype sequence may be included. Using a dPCR assay optical signals may be detected from the probes in each partition. The signals from the wildtype and variant sequence probes are used to generate a two-color plot. Subsequently, the presence of one or more variant sequences may be identified based on a deviation in the plotted signal from an expected wild-type cluster plot. This deviation may be a recognized pattern, characteristic of a particular variant sequence, whether in a subject or historically associated with a particular disease or condition.

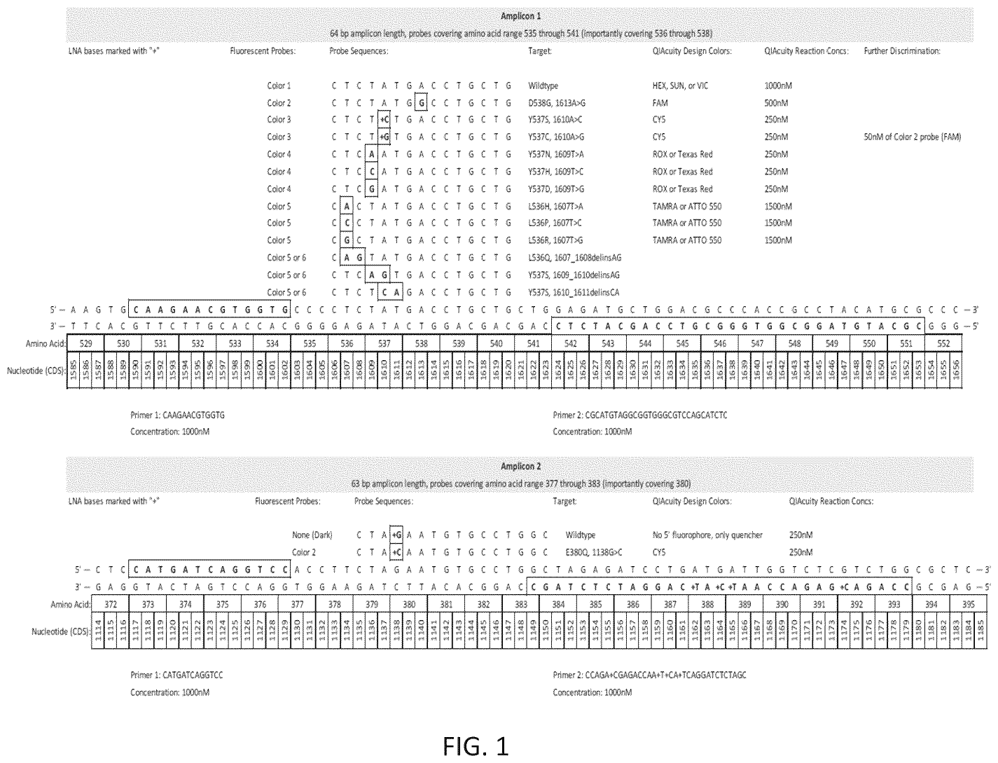

FIG. 1 provides an overview for exemplary sets of probes, including a wildtype probe, used to detect variant estrogen receptor 1 (ESR1) sequences in a sample obtained from a subject. The ESR1 protein regulates the transcription of many estrogen-inducible genes known to contribute to functions of growth, metabolism, sexual development, gestation, and other reproductive functions. Critically, ESR1 transcript variants are a hallmark in certain forms of breast cancer. Among these variants are recurrent variant ESR1 sequences, which are frequently found in patients newly diagnosed with metastatic and loco-regional recurrence of endocrine-treated breast cancer.

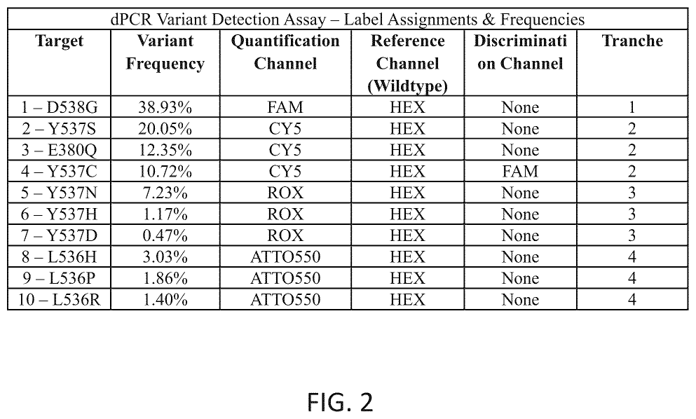

FIG. 2 summarizes probe sets from FIG. 1. As shown in FIG. 2, ten unique probes were created for variant ESR1 sequences in addition to a labeled, wildtype probe. The column labeled “Target” identifies the particular ESR1 variant sequence targeted by each probe, which is indicated using the resulting amino acid mutation relative to the wildtype ESR1 sequence. In this case, the relative frequencies of the variant sequences are known, however, methods of the invention may be used or include steps to elucidate the variant sequence frequencies. The “Quantification Channel” refers to the optical label, and thus, optical channel used to detect the presence of a labeled probe in a dPCR petition. The “Reference Channel” indicates the label, and corresponding optical channel, used to detect the wildtype sequence probe in the reaction partitions. The “Discrimination Channel” refers to a probe for one of the variant sequences in which an additional optical label is used, as will be described in greater detail, for further discriminating a variant sequence. Finally, as shown, the variants are separated into four different tranches, each using the same optical label. Tranche 1 includes the most prevalent mutant sequence. The remaining tranches contain variant sequences with fairly similar frequencies and/or corresponding genomic locations of the mutation giving rise to the variant.

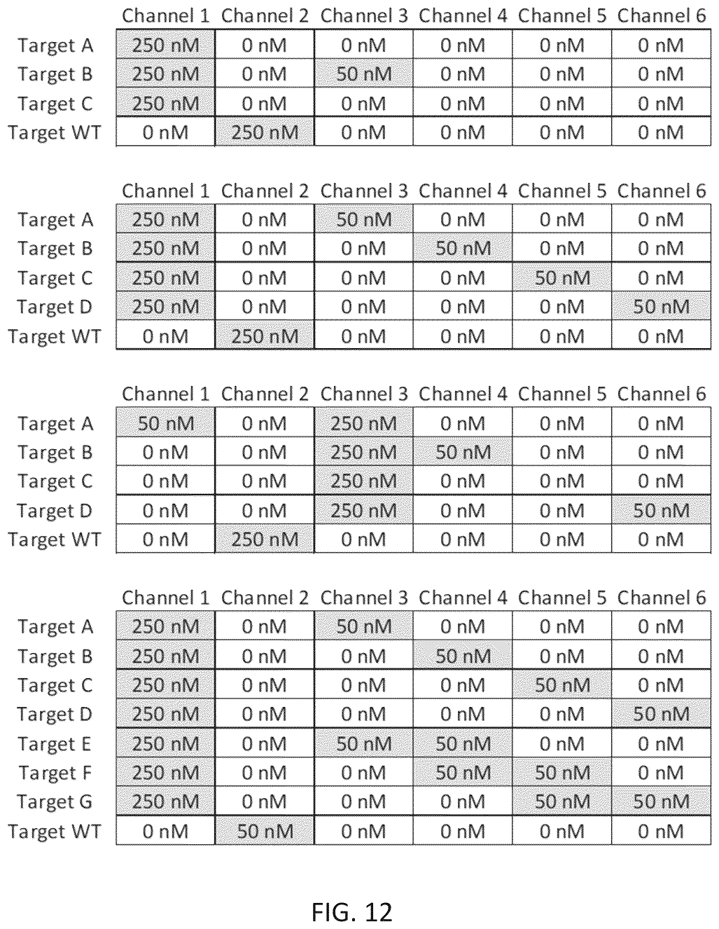

FIG. 12 shows probe reaction concentrations for example assay designs. Targets are detected in channels with target specific probes at higher concentrations (e.g., 250 nM) and discrimination is facilitated with target specific probes at lower concentrations (e.g., 50 nM).

In practice, methods of the invention may include the steps of: (i) providing a sample comprising one or more target nucleic acids, in this case ESR1 sequences; (ii) optionally, performing a pre-amplification such as a variant enrichment sample preparation reaction to selectively increase copy number of the target nucleic acid in the sample, in this case using the primers for each amplicon described in FIG. 1; (iii) aliquoting the sample into a plurality of subsamples, each in its own partition with the requisite set or sets of probes; (iv) conducting dPCR reaction on the subsamples; and (v) detecting the resulting optical signals from the probes, fluorescent probes such as hydrolysis probes that show the presence of a wildtype or variant sequence being produced in each partition. The optical signals form each partition are then detected and plotted to identify the presence or absence of the interrogated variant sequences.

In the exemplary assay outlined in FIGS. 1-2, the most common mutation, p.D538G is detected based on its optical label using a dedicated channel (FAM/Green). The next three most common mutations (p.Y537S, p.E380Q, and p.Y537N) are detected based on the fluorophore for that tranche of probes/variants using CY5/Crimson channel. In certain methods of the invention, and as exemplified for variant p.Y537C, a second probe is used with a different optical label, in this case, FAM detected by the Green channel. Although a discriminating probe is not necessarily required, even when used, the variants may still be identified simultaneously using less optical labels/channels than variants detected. Continuing, in the ROX/Red channel, a tranche of three (3) less frequent mutations (p.Y537N, p.Y537H, and p.Y537D) are detected and in the fourth tranche three (3) less frequent mutations are detected in the ATTO550/Orange channel (p.L536H, p.L536P, and p.L536R).

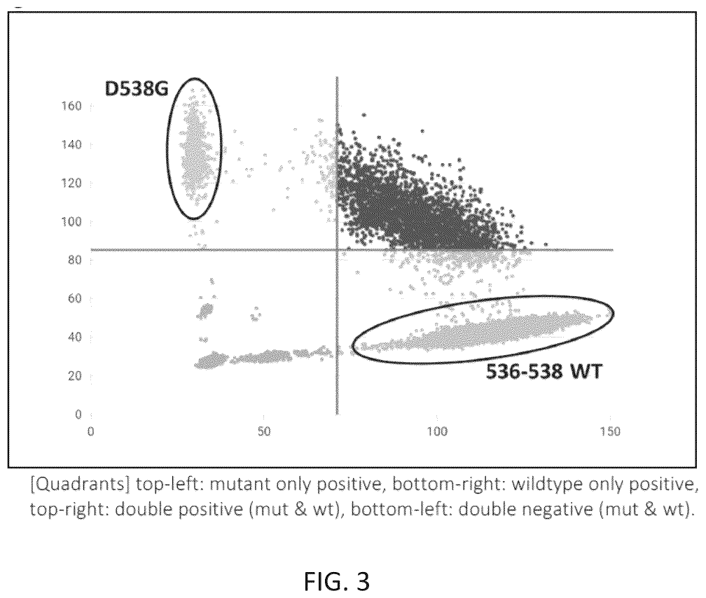

FIG. 3 provides a resulting two-color plot for the p.D538G mutation (using the D538G FAM/Green channel) against the wildtype probe signals (Hex/Yellow channel) for the dPCR reaction. In the plot, the Y-axis is the FAM/Green channel (variant) probe fluorescence intensity, and the X-axis is the HEX/Yellow channel (wildtype) fluorescence intensity. The two-color plot is divided into quadrants, in which: top-left is variant probe signal positive only positive; bottom-right is wildtype only positive; top-right is double positive (variant & wildtype); and the bottom-left is double negative (variant & wildtype). In certain aspects, as was done here, it is advantageous to dedicate a channel to the most frequently occurring variant to maximize sensitivity and provide an internal validation of the assay. As shown, the signals from the FAM/Green channel from the D538G probes produce a clearly identifiable pattern when plotted against the HEX/Yellow channel, which indicates the presence of the variant in the sample.

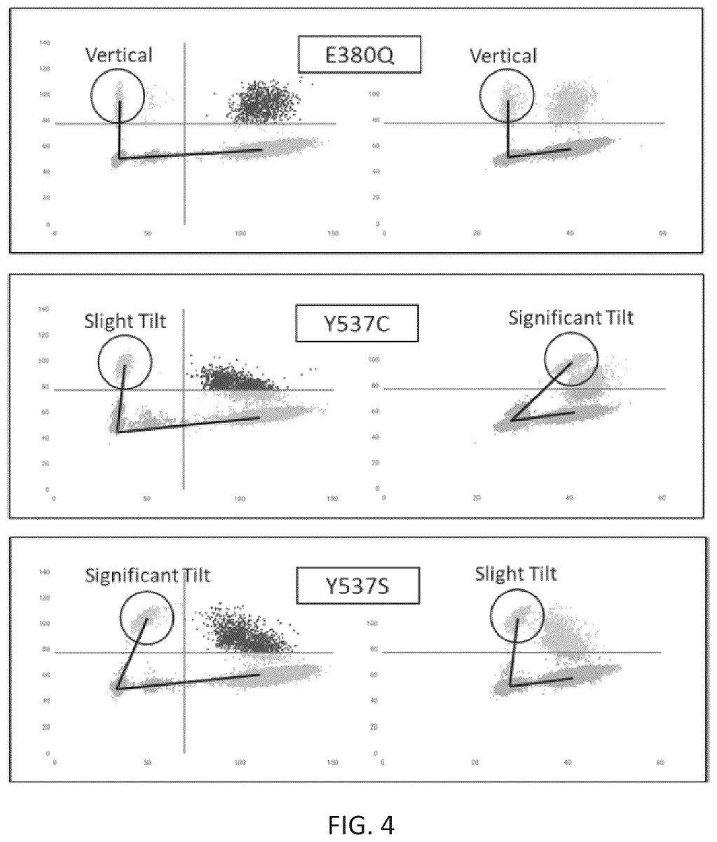

FIG. 4 provides similar plots for the tranche of variants containing variants p.Y537S, p.E380Q, and p.Y537N that are detected based on their CY5 probes using the Crimson channel. In the left plot for each variant, the X-axis represents the wildtype probe signal (HEX/Yellow) and the Y-axis represents the variant probe signals (CY5/Crimson). As shown, when plotted, each variant produces a signature pattern in its deviation relative to the wildtype plot—vertical for p.E380Q, slightly tilted for Y537C. and significantly tilted for Y537S. As described in FIGS. 1-2, included in this assay was a small amount of the Y537H probe, which had a FAM label detectable using the Green channel. The righthand plot for each variant shows the results of plotting the Green channel signals versus the Crimson channel signals. As shown, the deviation from the wildtype plot changes when these channels are interrogated, which helps discriminate the presence of the Y537C and Y537H probes.

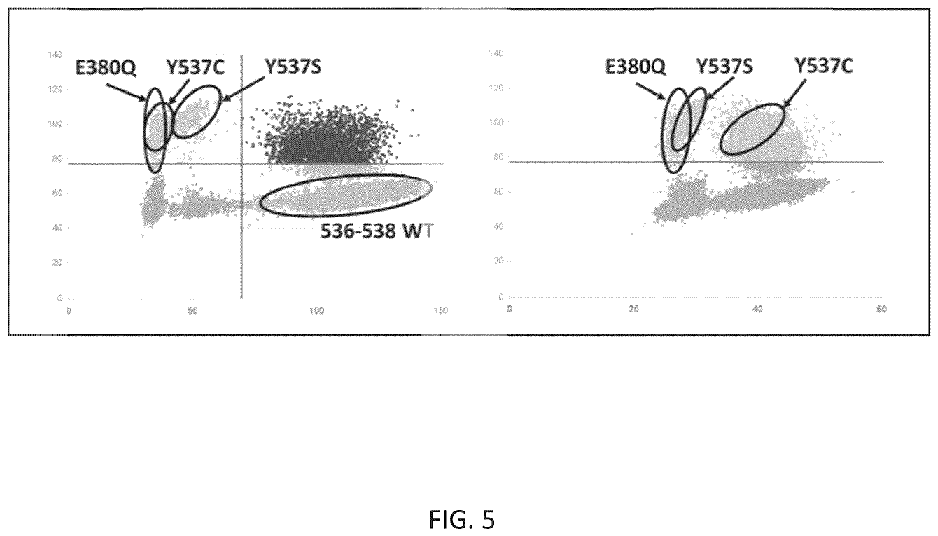

FIG. 5 provides a composite of both the right and left plots of FIG. 4. As shown, each mutation provides a characteristic pattern or fingerprint relative to the wildtype signals, which may be used to identify the presence or absence of each variant sequence in the sample.

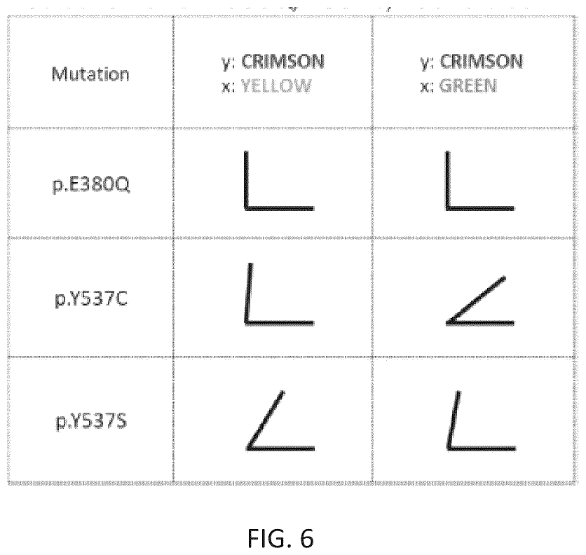

FIG. 6 provides schema for each variant's distinct fingerprints/patterns when interrogated using probes as described herein. Thus, methods of the invention may be used not only to detect variant sequences based on their characteristic dPCR pattern/fingerprint relative to the wildtype probe signal, but also used to discern the patterns/fingerprints, whether for an individual subject or for a population-wide assay.

FIG. 7 provides a composite of the dPCR signals plotted for the variants in the tranche containing Y537D, Y537N, and Y537H using the ROX/Red channel corresponding to the variant probes and HEX/Yellow for the wildtype. The sum of the three variant frequencies is estimated to be 8.7%. Signal quality of the two more common variants (Y537N and Y537H, 8.2% combined) is readily detected. Further, the signal amplitude for Y537D, which is extremely rare (0.47%) is low, but also detectable.

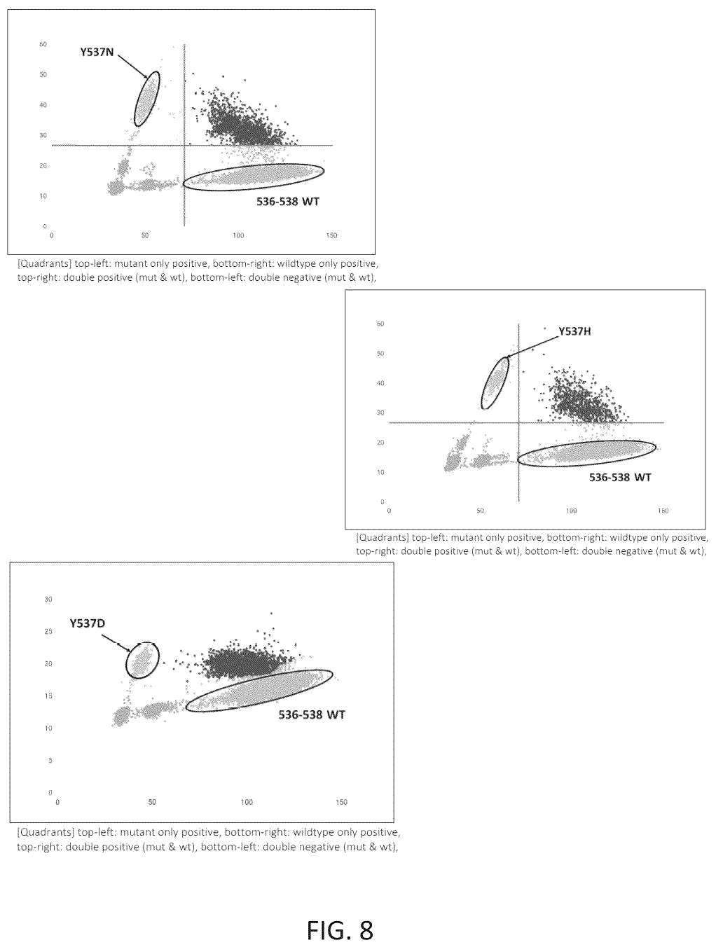

FIG. 8 provides the individual plots for the Y537D, Y537N, and Y537H variants, which were used to discern the patterns/fingerprints for each variant.

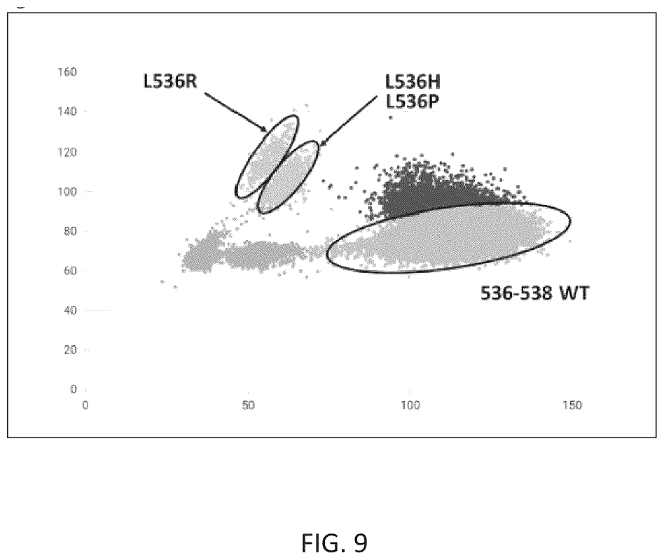

FIG. 9 provides a composite of the dPCR signals plotted for the variants in the tranche containing L536R, L536H, and L536P using the ATTO550/Orange channel corresponding to the variant probes and HEX/Yellow for the wildtype. As shown, a pattern/fingerprint is clearly evident for each of these rare variant sequences when plotted against the wildtype channel.

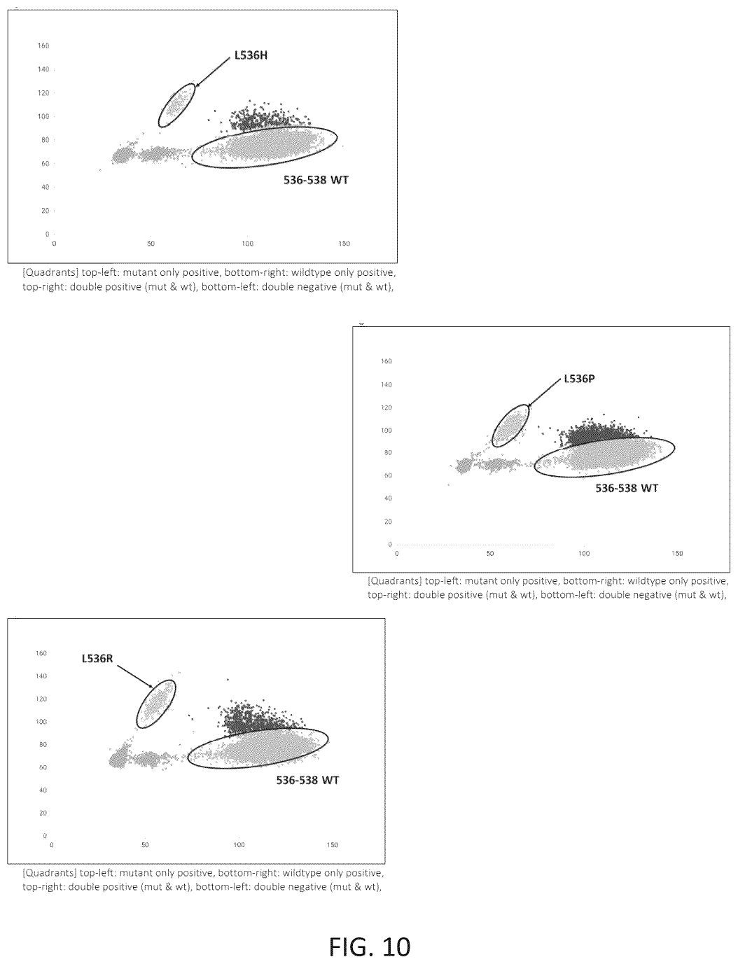

FIG. 10 provides the individual plots for the L536R, L536H, and L536P variants, which were used to discern the patterns/fingerprints for each variant.

Methods of the present invention are useful for detecting whether variant sequences are present in a sample, which may comprise a mixture of target nucleic acids wherein only a portion of the target nucleic acids may comprise the variant sequences. In particular, the methods are useful for detecting the presence of a variant sequence in a sample comprising target nucleic acids of which only a minor fraction may potentially comprise the variant sequence.

The target nucleic acid sequence may be any target nucleic acid sequence, which is desirable to detect. However, in preferred aspects, the target nucleic acid sequence is a variant sequence associated with a clinical condition, e.g., cancer.

In certain methods of the invention detecting the presence of variant sequences, the methods may include providing a sample comprising one or more target nucleic acids, and performing pre-amplification to increase copy number of the target nucleic acid in the sample. Subsequently, the pre-amplified sample is aliquoted into a plurality of subsamples. The method may then include conducting a PCR (preferably dPCR) on the subsamples, and detecting the target nucleic acids, e.g., variant sequences.

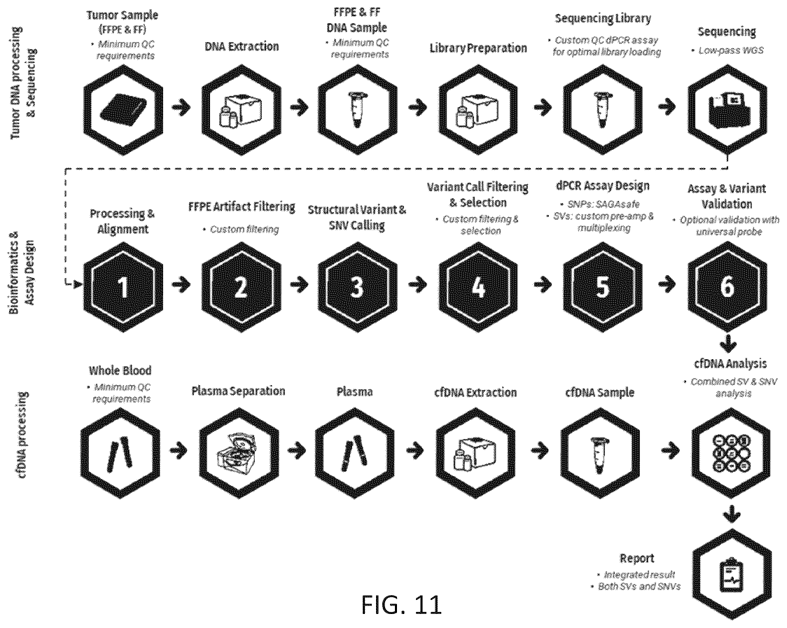

FIG. 11 provides an exemplary workflow showing sample acquisition, bioinformatics workflow and sample processing to detect and/or identify variant sequences. This exemplary workflow shows low pass whole genome sequencing of the tumor as a first step, followed by filtering and selection of the target, which may be known to be the location of sequence variants, e.g., structural variants or a mutation (or the combination thereof).

Methods of the invention may comprise the use of an incremental nucleic acid amplification step as a targeted pre-amplification, i.e., to increase relative abundance of a target within a sample. In some embodiments, preamplification is exponential and increases abundance of select targets. The use of targeted pre-amplification increases the number of copies of the target nucleic acids in the sample. The pre-amplification could be symmetrical or asymmetrical, or a combination of the two. In an asymmetrical or incremental amplification, a single strand of nucleic acids is linearly copied, preferentially without copying other strands among the sample. Some embodiments perform incremental amplification by providing only one primer that copies a target (instead of the forward- and reverse-primer pair of PCR). Certain embodiments discussed herein perform the asymmetrical amplification using a primer-H and a primer-L. See also U.S. Pat. No. 11,066,707 and U.S. Pub. 2022/0056533 A1, both incorporated by reference.

Methods of the invention have a very low limit of detection. This enables a detection of target variant sequences that are often present at very low levels, and/or detection of the presence of variant sequences potentially present at very low levels in mixtures comprising other target nucleic acid sequences.

### Sample and Target Nucleic Acids

The sample may be any sample in which it is desirable to detect whether said variant sequence is present. For example, if the variant sequence is indicative of a clinical condition, the sample may be a sample from an individual at risk of acquiring said clinical condition. The variant sequences may differ from the wild-type sequence by substitution(s), deletion(s) and/or insertions(s).

Methods of the invention further provide that the one or more targeted nucleic acid molecules are associated with a variant sequence, wherein the variant sequence may be a variant of a wildtype nucleic acid sequence. The variant sequence may be selected from the group consisting of single nucleotide variants (SNVs), insertions and deletions (indels), duplications, copy-number variants (CNVs), inversions, and translocations. The methods of the invention may also be used to analyze the cfDNA/ctDNA samples from a cancer patient, optionally including mutant or structural variants from cfDNA/ctDNA.

In some embodiments, methods of the invention include one or more steps that diagnoses a subject based on the detection of one or more variant sequences. Such a diagnosing step may, for example diagnose a subject with a disease associated with a detected variant sequence, reporting a likelihood that the patient has or will develop such disease based on the identification of one or more variant sequence, and assessing the relative frequencies of identified variants to assess disease progression or disease response to a treatment.

Variant sequences identified using methods of the invention may be variants associated with a particular type or stage of cancer, or of cancer having a particular characteristic (e.g., metastasized, drug resistant, and drug responsive). For example, certain mutations are known to be associated with patient outcomes and/or specific conditions. In certain aspects, methods of the invention may provide information used in therapeutic decisions, guidance and monitoring, as well as development and clinical trials of therapies for a particular condition. For example, treatment efficacy can be monitored by comparing detected variants and/or variant frequencies from before, during, and after treatment. Longitudinal monitoring may be used to assess increases or decreases in variant sequences or frequencies, identify new or absent variant sequences after treatment, which may, for example, guide subsequent treatment decisions. In certain aspects, diagnosing a subject includes diagnosing the subject with a particular stage or type of cancer associated with a detected sequence variant.

For polymorphisms or small indels (typically <about 50 bases), it may be preferred that both the wild-type sequence and the variant sequence be amplified in a polymerase reaction by the pair of primers specifically capable of amplification of the target nucleic acid sequence. It may be preferred that the wild-type sequence and the variant sequence does not differ too much from each other in length. For structural rearrangements (typically at least about 50 bases), the variant is amplified by an incremental step prior to digital PCR and assays also, in parallel, include a reference assay targeting a stable region of the genome to quantify the signal. Structural variants are described in Mahmoud, 2019, Structural variant calling: the long and the short of it, Genome Biology 20:a246, incorporated by reference.

The sample may be any biological sample, including a bodily fluid sample comprises bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, or vomit.

### Amplification and Pre-Amplification

Methods of the current invention may include the use of amplification reactions. Amplification of a nucleic acid is the generation of copies of said nucleic acid.

Methods of the invention also include a pre-amplification step. The pre-amplification may be described as a variant enrichment sample preparation reaction step. Such a pre-amplification may amplify only select targets by a limited, controlled, or known amount. For example, ten cycle of linear pre-amplification with a primer specific for a variant would increase abundance of copies of that variant in the sample about tenfold. Because the abundance is increased by a known amount, if sequences in the reaction mixture are later quantified (e.g., by digital PCR), the quantities of those sequences in the original sample can be determined (using the know amount by which abundance of the select variants was increased). Additionally, the pre-amplification greatly increases the probability of detection of those variants, which aids in detection of very rare sequences in a sample. That is why it may be preferable to have the pre-amplification specifically increase the abundance of copies of the selected variant of the sample by known amounts. According to the invention, the pre-amplification step is a sample preparation to preferentially increase the number of copies of the selected targets through asymmetric incremental amplification or symmetric exponential amplification or a combination of the two. An incremental pre-amplification could be expected to proceed without more than linear copy amplification, compared to the exponential production of amplicons in PCR. The incremental amplification may proceed using un-paired primers (e.g., single primers) that get extended through, and copy, a target of interest. In some embodiments, an incremental pre-amplification comprises the use of at least a couple of primers, wherein only one primer is active in the pre-amplification. The said active primer may be active due to a difference in the melting temperature (Tm) and annealing temperatures of the primers. The use of these primers for asymmetrical incremental amplification is disclosed in U.S. Pat. No. 11,066,707, which is hereby incorporated by reference in its entirety.

Methods of the invention may use an incremental pre-amplification to increase the abundance of a target of interest in a sample, even when that target is present in very low numbers. This may alleviate problems with molecular detection assays that rely on PCR to amplify target, in which due to the stochastic nature of PCR, targets present only in very low numbers may go undetected and also in which PCR reactions are plagued by “dead volumes” that go undetected. Those problems are addressed by selectively increasing the abundance of a target of interest in what is described here as a pre-amplification step. In one embodiment, the pre-amplification step is not-exponential, i.e., is not PCR. Instead, the pre-amplification step is preferably incremental which may be taken to mean that extension products from one round of pre-amplification are not substrates for copying by any reverse primer. Rare targets of interest are increased in abundance by this pre-amplification step. The increase in abundance is approximately linear (not exponential) over a cycle of pre-amplification.

It may be preferable to perform incremental pre-amplification (and not exponential) so that other material in the sample is also still present an accessible to subsequent amplification steps. Preferred embodiments use pre-amplification that is specific to a genetic sequence selected for clinical significance such as a structural variant specific to nucleic acid from a tumor. In fact, methods of the invention may identify tumor mutations and select one or more structural variants for clinical significance, such as a structural variant that is likely to persist, e.g., even after cancer treatment such as chemotherapy.

Preferably, in methods of the invention, a sequence containing a possible variant is selected and then pre-amplified. The pre-amplification specifically increases the abundance of copies of the selected variant of the sample. The sample can then be assayed using the digital PCR (dPCR) assay, as described above, for the selected variant as well for any other variants potentially present in the sample. In fact, embodiments of the invention may be multiplexed using, e.g., differently labeled fluorescent hydrolysis probes to interrogate the sample for multiple variants simultaneously, while always using fewer distinct fluorescent reporters than variants detected. Because the selected variant was increased in abundance by the incremental pre-amplification, it will not be lost to dead volume or the stochastic nature of PCR. Thus, assays of the invention are useful for multiplexed detection of very rare targets in a sample, and may be particularly useful for detecting cancer-specific variant sequences such as in circulating tumor DNA (ctDNA) in a blood or plasma sample, such as in a liquid biopsy. Moreover, as discussed further herein, the incremental preamplification and the exponential amplification of dPCR can proceed in the presence of the same set of reagents (primers, dNTPs, polymerase, ions, probes, etc.) without requiring a clean-up step, or a reagent change, or the addition of reagents as the assay progresses. In fact, embodiments discussed herein use a primer pair that functions to incrementally pre-amplify a target under one thermocycle and exponentially amplify the target for dPCR under a different thermocycle.

In methods of the invention nucleic acid polymerase enzymes used may have different elongation temperatures. The elongation temperature is a temperature allowing enzymatic activity of the nucleic acid polymerase following primer annealing. Typically, a nucleic acid polymerase has activity over a temperature range, and thus the elongation temperature may be any temperature within that range. Most nucleic acid polymerases have a temperature optimum but retain activity at other temperatures than the temperature optimum. In such cases, the elongation temperature may be any temperature allowing the primer to anneal and the nucleic acid polymerase has activity even if the temperature is not the optimum temperature. At the elongation temperature of a nucleic acid polymerase, the enzyme is capable of catalyzing synthesis a new nucleic acid strand complementary to the template strand at the elongation temperature. In certain methods, the elongation temperature is near the melting temperature of the primer-H. Thus, a nucleic acid polymerase may be chosen, which has polymerase activity at a temperature near the melting temperature of primer-H and/or the primer-H may be designed to have a melting temperature near the elongation temperature.

The amplicons or product produced during a pre-amplification step such as a variant enrichment sample preparation reaction step may be used as direct sample input for the PCR. That is, after the pre-amplification, reagents for PCR may be added to the reaction mixture from the pre-amplification. For example, if the pre-amplification is performed in a tube, after pre-amplification, the tube can be topped-up (without clean-up) to add any additional reagents useful in PCR. In some embodiments, primers used in the invention are a part of a plurality of pairs of primers capable of amplification of different target nucleic acid sequences. Pre-amplification can be performed under a first temperature control then, after optionally adding any further reagents to the tube and optionally without any clean-up, PCR may be performed under a second temperature control. Methods of the invention may be performed without any cleanup steps, which could lead to loss of analyte material.

Methods of the invention may involve the use of PCR reagents for the sample preparation reactions and the dPCR. The sample preparation reaction and/or dPCR may include at least part of the sample, the set of primers, and sufficient PCR reagents to allow a polymerase reaction. Methods and reagents useful for performing a PCR reaction are well known to the skilled person. For example, the PCR reaction may comprise any of the nucleic acid polymerase and PCR reagents, described herein below in the section “PCR reagents”. Depending on the mode of detecting whether the sample preparation product comprises the variant sequence, the sample preparation reaction may also comprise detection reagents. PCR reagents are reagents which are added to a PCR in addition to nucleic acid polymerase, sample and set of primers. The PCR reagents at least comprise nucleotides. In additional the PCR reagents may comprise other compounds such as salt(s) and buffer(s).

For most purposes the PCR reagents comprise nucleotides. Thus, the PCR reagents may comprise deoxynucleoside triphosphates (dNTPs), in particular all of the four naturally-occurring deoxynucleoside triphosphates (dNTPs).

The PCR reagents frequently comprise deoxyribonucleoside triphosphate molecules, including dATP, dCTP, dGTP, dTTP. In some cases, dUTP is added.

The PCR reagents may also comprise compounds useful in assisting the activity of the nucleic acid polymerase. Thus, the PCR reagent may comprise a divalent cation, e.g., magnesium ions. Said magnesium ions may be added on the form of e.g., magnesium chloride or magnesium acetate (MgCl2) or magnesium sulfate is used.

The PCR reagents may also comprise one or more of the following:
non-specific blocking agents such as BSA or gelatin from bovine skin, betalactoglobulin, casein, dry milk, or other common blocking agents,
non-specific background/blocking nucleic acids (e.g., salmon sperm DNA),
biopreservatives (e.g., sodium azide),
PCR enhancers (e.g., Betaine, Trehalose, etc.),
inhibitors (e.g., RNAse inhibitors).

The PCR reagent a may also contain other additives, e.g., dimethyl sulfoxide (DMSO), glycerol, betaine (mono)hydrate (N,N,N-trimethylglycine=[caroxy-methyl]trimethylammonium), trehalose, 7-Deaza-2′-deoxyguanosine triphosphate (dC7GTP or 7-deaza-2′-dGTP), formamide (methanamide), tetramethylammonium chloride (TMAC), other tetraalkylammonium derivatives (e.g., tetraethylammonium chloride (TEA-Cl) and tetrapropylammonium chloride (TPrA-CI), non-ionic detergent (e.g., Triton X-100, Tween 20, Nonidet P-40 (NP-40)), or PREXCEL-Q.

The PCR reagents may comprise a buffering agent.

In some cases, a non-ionic Ethylene Oxide/Propylene Oxide block copolymer is added to the aqueous phase in a concentration of about 0.1%, 0.2%, 0.3%, 0.4%, 0.5%, 0.6%, 0.7%, 0.8%, 0.9%, or 1.0%. Common biosurfactants include non-ionic surfactants such as Pluronic F-68, Tetronics, Zonyl FSN. Pluronic F-68 can be present at a concentration of about 0.5% w/v.

A wide range of common, commercial PCR buffers from varied vendors can be substituted for the buffered solution.

Methods of the invention generally include the use of a nucleic acid polymerase. The nucleic acid polymerase may be any nucleic acid polymerase, such as a DNA polymerase. The nucleic acid polymerase should have activity at the elongation temperature.

The nucleic acid polymerase may be a DNA polymerase with 5′ to 3′ exonuclease activity. This may in particular be the case in the methods of the invention, wherein the methods or kits involves use of a detection probe, such as a Taqman detection probe.

Any DNA polymerase, e.g., a DNA polymerase with 5′ to 3′ exonuclease activity that catalyzes primer extension can be used. For example, a thermostable DNA polymerase can be used. Preferably, the nucleic acid polymerase is a Taq polymerase, enabling the use of Taqman probes to identify variant sequences as described herein.

Methods of the invention include a sample preparation reaction step, which could be either asymmetric incremental amplification or symmetrical exponential amplification or a combination of the two. Asymmetric incremental amplification may proceed using unpaired, e.g., single primers (even if multiple primers are used, each may be “single” in the sense of not being paired with a reverse primer that anneals to the extension product of the single primer). In some embodiments, incremental, pre-amplification may include the use of at least two primers, wherein only one primer is active in the sample preparation reaction. For example, the said active primer is active due to a difference in the melting temperature (Tm) and annealing temperatures of the provided primers.

More preferably, the step of asymmetrical incremental amplification includes: (i) providing a pair of primers capable of amplification of a target nucleic acid, wherein the pair of primers comprises a primer-H and a primer-L. According to the methods of the invention, the melting temperature of primer-H may be about 10° C. to about 22° C. higher than the melting temperature of primer-L, and primer-L comprises a sequence complementary to a fragment of the elongation product of primer-H, and the sample preparation reaction is performed. The sample preparation reactions also include nucleic acid polymerase having polymerase activity, primer(s), and PCR reagents.

In the embodiments with primer-H and primer-L, the incremental preamplification will proceed using an annealing temperature at which primer-H (but not primer-L) anneals. During that incremental amplification, primer-H will function as a “single” primer (despite the presence of primer-L). Primer-L will not anneal to anything to any meaningful extent because the reaction mixture is not brought down to the annealing temperature of primer-L. The incremental preamplification may be run for a predetermined number of cycles (e.g., one, or five, etc.) or for a fixed amount of time, or until the sample exhibits a result (change in optical density, cleavage of a fluorescent probe, etc.). After the incremental preamplification, then the reaction mixture may be subject to exponential amplification. For the exponential amplification, at the annealing step, the temperature is brought down to the annealing temperature of primer-L, which promotes annealing of both primer-H and primer-L.

It is important to note that preceding paragraphs describes the function of primer-H and primer-L as those primers may function among many other primers. For example, tens, hundreds, or thousands, or more loci can be probed in parallel using a corresponding number of primer pairs. For an example, in a particular assay, 24 loci are being probed in parallel (but that number 24 is arbitrary, and could just as easily be 1, or 2, or 3, or 6, or 17, or 96, or 99, or 384, or 1,000, or 1,536, or an integer multiple of any of those numbers, etc.) An assay of the invention may use a primer pair for each loci, e.g., may use 24 primer pairs. Any one or any number of those primer pairs may fit the description of primer-H and primer-L. However, and this is important, it may be desirable to pre-amplify only certain loci, so any number of the primer pairs may include forward and reverse primer that each have an annealing temperature essentially the same as for a primer-L.

In other embodiments, the pre-amplification proceeds with unpaired “single” primers (e.g., that operate at the primer-H annealing temperature. After that, the reaction mixture (original sample, plus added reagents, plus preamplification product) may be subject to conditions for exponential amplification. The exponential amplification may use primer pairs, any one of which may “match” all or part of the single primer, or than anneal to targets within the length of the extension product of the unpaired single primers. Those reactions may proceed at different temperatures, or the paired primers may not be made available until after the incremental preamplification. For example, the paired primers may be added (e.g., by microfluidic handling) or released from confinement or attachment (e.g., by chemical, temperature, or photo lysis of a hydrogel bead).

In certain methods of the invention, an asymmetrical incremental amplification may be used, which includes: providing a pair of primers capable of amplification of a target nucleic acid, wherein the pair of primers comprises a primer-H and a primer-L, wherein the melting temperature of primer-H is about 10° C. to about 22° C. higher than the melting temperature of primer-L, and wherein primer-L comprises a sequence complementary to a fragment of the elongation product of primer-H; providing a nucleic acid polymerase having polymerase activity at an elongation temperature; and preparing sample preparation reactions, each comprising a part of the sample, the set of primers, the nucleic acid polymerase, and PCR reagents. The melting temperature of primer-H may be about 10° C. higher than the melting temperature of primer-L. Preferably, the melting temperature of primer-H is about 12° C. higher than the melting temperature of primer-L. More preferably, the melting temperature of primer-H is about 16° C. higher than the melting temperature of primer-L.

In certain methods, an asymmetric incremental amplification may also include a set of primers, wherein at least one primer is specifically capable of amplification of only one strand of the target nucleic acid sequence, and performing the sample preparation reaction in a solution including a nucleic acid polymerase having polymerase activity, and the sample preparation reaction is performed. The sample preparation reactions also include nucleic acid polymerase having polymerase activity, primer(s), and PCR reagents.

The invention may further include a step of symmetric exponential amplification, wherein the symmetric exponential amplification include the steps of (i) providing a set of primers specifically capable of amplification of the target nucleic acid sequence; (ii) providing a nucleic acid polymerase having polymerase activity at an elongation temperature; (iii) preparing sample preparation reactions each comprising a part of the sample, the set of primers, the nucleic acid polymerase, and PCR reagents; and performing the sample preparation reaction.

Certain methods of the invention may include an asymmetrical incremental amplification followed by symmetrical exponential amplification. The steps involved in asymmetrical incremental amplification and symmetrical exponential amplification are outlined above.

Methods of the current invention may also include a symmetrical exponential amplification is followed by an asymmetrical incremental amplification. The steps involved in asymmetrical incremental amplification and symmetrical exponential amplification are outlined above.

Methods of the current invention may also include an asymmetric incremental amplification and said symmetric exponential amplification may be performed in the same reaction volume. In certain methods, the asymmetric incremental amplification and the symmetric exponential amplification may be performed using the same set of primers.

In certain methods of the invention an asymmetric incremental amplification may be activated at a higher temperature as compared to symmetric exponential amplification. For example, a thermocycler may be programmed to go down only to the higher annealing temperature for the incremental amplification but to go to a lower annealing temperature for exponential amplification.

Methods of the invention may include a step of conducting a plurality of PCR reactions on the sample, which has undergone the sample preparation reaction, wherein the step includes a pair of primers capable of specific amplification of a target nucleic acid, a nucleic acid polymerase having a polymerase activity at an elongation temperature, preparing PCR reactions, wherein each PCR reaction comprises a part of the sample, the set of primers, the nucleic acid polymerase, PCR reagents; and performing symmetrical exponential amplification. Preferably, these PCR reactions are conventional PCR reactions. Preferably, the PCR is digital PCR (dPCR), and uses variant sequence discriminating probes as described. The sample including the products following the sample preparation reaction step may be used as direct sample input for the PCR. Optionally, the pair of primers used in the invention are a part of a plurality of pairs of primers capable of amplification of different target nucleic acid sequences.

Methods of the invention may further include the use of a plurality of pairs of primers capable of amplification of different target nucleic acid sequences. Preferably, the methods of the invention include the use of multiplex PCR.

Methods of the invention may use quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCR-RFLP/RT-PCR-RFLP, hot start PCR, nested PCR, in situ polony PCR, in situ rolling circle amplification (RCA), digital PCR (dPCR), droplet digital PCR (ddPCR), bridge PCR, picotiter PCR, and emulsion PCR.

### Primer-H and Primer-L

Methods of the invention may include the use of several primers including primers named as primer-H and primer-L. Primer-H is a primer having a high melting temperature, whereas primer-L is a primer having a low melting temperature. The melting temperature of a primer is the temperature at which 50% of the primer forms a stable double helix with its complementary sequence and the other 50% is separated to single strand molecules. The melting temperature may also be referred to as Tm or Tm. Preferably, the Tm as used herein is calculated using a nearest-neighbor method based on the method described in Breslauer, 1986, Predicting DNA duplex stability from the base sequence, PNAS 83:3746-50 (incorporated by reference) using a salt concentration parameter of 50 mM and primer concentration of 900 nM. For example, the method is implemented by the software “Multiple Primer Analyzer” from Life Technologies/Thermo Fisher Scientific Inc.

Certain methods of the invention may use of a set of primers comprising a primer-H and a primer-L, wherein the melting temperature of primer-H is about 10° C. to about 22° C., preferably at least 10° C., more preferably at least 15° C. higher than the melting temperature of primer-L, and wherein primer-L contains a sequence complementary to the elongation product of primer-H.

The primer-H is preferably designed as a primer for amplification of the target sequence or the sequence complementary to the target sequence. Thus, the primer-H is preferably capable of annealing to either the target nucleic acid sequence or to the sequence complementary to the target nucleic acid sequence. For example, primer-H may be capable of annealing to the complementary strand of the target nucleic acid sequence at the 5′-end or close to the 5′-end of the target nucleic acid sequence, or the primer-H may be capable of annealing to the target nucleic acid sequence at the 3′-end or close to the 3′-end of the target nucleic acid sequence. Thus, the primer-H may comprise a sequence identical to the 5′-end of the target nucleic acid sequence. The primer-H may even consist of a sequence identical to the 5′-end of the target nucleic acid sequence. The primer-H may also comprise a sequence identical to the target nucleic acid sequence. Thus, the primer-H may comprise a sequence complementary to the 3′-end of the target nucleic acid sequence. The primer-H may even consist of a sequence complementary to the 3′-end of the target nucleic acid sequence.

Similarly, primer-L is preferably designed as a primer for amplification of the target sequence or the sequence complementary to the target sequence. If the primer-H is designed for amplification of the target sequence, the primer-L is preferably designed for amplification of the sequence complementary to the target sequence and vice versa. Thus, the primer-L is preferably capable of annealing to either the target nucleic acid sequence or to the sequence complementary to the target nucleic acid sequence. If primer-H is capable of annealing to the target nucleic acid sequence, then primer-L is preferably capable of annealing to the sequence complementary to the target nucleic acid sequence and vice versa. For example, primer-L may be capable of annealing to the complementary strand of the target nucleic acid sequence at the 5′-end or close to the 5′-end of the target nucleic acid sequence, or the primer-L may be capable of annealing to the target nucleic acid sequence at the 3′-end or close to the 3′-end of the target nucleic acid sequence. Thus, the primer-L may comprise a sequence identical to the 5′-end of the target nucleic acid sequence. The primer-L may even consist of a sequence identical to the 5′-end of the target nucleic acid sequence. The primer-L may also comprise a sequence identical to the target nucleic acid sequence. Thus, the primer-L may comprise a sequence complementary to the 3′-end of the target nucleic acid sequence. The primer-L may even consist of a sequence complementary to the 3′-end of the target nucleic acid sequence.

Primer-H may have a nucleotide sequence identical to the sequence at the 5′-end of the target nucleic acid sequence and the primer-L comprises or consists of a sequence identical to the complementary sequence of the 3′-end of the target nucleic acid sequence.

Primer-L may have a nucleotide sequence identical to the sequence at the 5′-end of the target nucleic acid sequence and the primer-H comprises or consists of a sequence identical to the complementary sequence of the 3′-end of the target nucleic acid sequence.

Primer-H and primer-L are designed to have the melting temperatures as indicated herein. The skilled person will be capable of designing primer-H and primer-L to have the desired melting temperature by adjusting the sequence of the primers, the length of the primers and optionally by incorporating nucleotide analogues as described herein above in the section “Set of primers”.

Primer-H is designed so that it has an annealing temperature which is significantly higher than the annealing temperature of primer-L, for example at least 10° C. higher. Thus, the melting temperature of primer-H may be at least 12° C. higher, for example at least 15° C. higher, preferably at least 14° C. higher, even more preferably at least 16° C. higher, yet more preferably 18° C. higher, such as at least 20° C. higher, for example in the range of 15 to 50° C., such as in the range of 15 to 40° C., for example in the range of 15 to 25° C. higher than the melting temperature of primer-L.

In general, it may be preferred that the melting temperature of primer-H is as high as possible, but not higher than the highest functional elongation temperature of at least one nucleic acid polymerase. Said elongation temperature does not need to be the optimum temperature for said nucleic acid polymerase, but it is preferred that at least one nucleic acid polymerase has activity at the melting temperature primer-H. Thus, the melting temperature of the primer-H may approach or may even exceed 80° C.

Since it is also preferred that the melting temperature of primer-L is sufficiently high to ensure specific annealing of primer-L to the target nucleic acid sequence/the complementary sequence of the target nucleic acid sequence, and the melting temperature of primer-H should be significantly higher than the melting temperature of primer-H, then frequently, the melting temperature of primer-H is at least 60° C. The melting temperature of primer-H may also frequently be at least 70° C. The melting temperature of primer-H may for example be in the range of 60 to 90° C., for example in the range of 60 to 85° C., such as in the range of 70 to 85° C., for example in the range of 70 to 80° C.

The melting temperature of primer-L is preferably sufficiently high to ensure specific annealing of primer-L to the target nucleic acid sequence/the complementary sequence of the target nucleic acid sequence, but also significantly lower than the melting temperature of primer-H. Frequently, the melting temperature of the primer-L is in the range of 30 to 55° C., such as in the range of 35 to 55° C., preferably in the range of 40 to 50° C.

Methods of the invention may also include the use of a set primers or a plurality of primers. A set of primers or a plurality of primers contain two or more different primers. A set of primers contains at least a pair of primers specifically capable of amplification of a target nucleic acid. Furthermore, a set of primers according to the invention contains at least a primer-H and a primer-L. Thus, wherein the set of primers contains only two different primers, then set of primers contains a primer-H and a primer-L, wherein the primer-H and primer-L are capable of amplification of a target nucleic acid.

### Detection

The invention in general comprises a step of detecting, whether the sample comprises the variant sequences. Said detection may be accomplished in any suitable manner known to the skilled person. For example numerous useful detection methods are known in the prior art, which can be employed with the methods of the invention.

The step of detection may include the presence of a detection reagent in PCR reactions. Said detection reagent may be any detectable reagent, for example it may be a compound comprising a detectable label, wherein said detectable label for example may be a dye, a radioactive activity, a fluorophore, a heavy metal or any other detectable label.

Frequently the detection reagent comprises a fluorescent compound associated with a variant-sequence specific probe as described herein.

The detection reagent may include detection probes. Detection probes may include nucleotide oligomers or polymers, which optionally may comprise nucleotide analogues. Frequently, the detection probe may be a DNA oligomer. Typically, the detection probe is linked to a detectable label, for example by a covalent bond. The detectable label may be any of the aforementioned detectable labels, but preferably is an optically detectable label such as a fluorophore.

The detection probe is in general capable of specifically binding the target nucleic acid sequence. For example, the detection probe may be capable of specifically binding the target nucleic acid comprising a variant sequence. Thus, the detection probe may be capable of annealing to the target nucleic acid sequence or to the sequence complementary to the target nucleic acid sequence. Thus, the detection probe may comprise a sequence identical to a fragment of the target nucleic acid sequence or the sequence complementary to the target nucleic acid sequence. It is generally preferred that the detection probe comprises a sequence different to the sequence of any of the primers of the set of primers.

### Quantification

Methods of the invention may include steps of providing a sample comprising one or more target nucleic acids; performing sample preparation reaction to increase copy number of the target nucleic acid in the sample; aliquoting the sample into a plurality of subsamples; conducting polymerase chain reaction (PCR) on the subsamples; and detecting variant nucleic acid sequences. Using targeted pre-amplification, an increased number of copies of the target is available may be available for subsequent PCR reactions used to identify the variant sequences. An advantage of the pre-amplification is that it significantly reduces or eliminates the instance of false-negatives, which is often an issue in samples with low concentration of target, by reducing the problem of dead volumes and stochastic sampling error.

The invention also provides a reference assay for detection of structural variants or mutants of patient's cfDNA. In particular, the invention provides a method for detecting wildtype cfDNA and any variant sequences of interest included within that cfDNA. Accordingly, the invention provides an assay, comprising the use of a pair of primers and probe, to detect and/or quantify a wildtype (reference) sequence of cfDNA. The invention further provides an assay to calculate variant allele fraction (VAF) for the target nucleic acids in the sample. In certain aspects, a target nucleic acid may be a variant of the wild-type nucleic acid sequence in the sample.

Certain methods of the invention may further provide the use of publicly available information to determine the regions of the genome that are stable for amplification and design the assay accordingly. As an example, a person of ordinary skill in the art may determine that chromosome 2, band 13 (2p13) is stable, and consequently, less susceptible to changes in copy numbers. The invention further provides that this information may be used to design the assays, especially to minimize the variations in copy numbers in the sample.

Methods of the invention may include a step that quantifies the amount of the target nucleic acid present in the sample. The replication assays have variability in the pre-amplification step. In particular, the efficiency of pre-amplification may be less than 100%. Thus, a correction factor is applied to back-calculate original sample concentrations. As an example of this issue, the pre-amplification step may result in 50-fold amplification in one instance but can only generate 30-fold amplification in another instance, resulting in 50-fold or 30-fold copies of the target sample respectively. Moreover, denaturation state of the sample also impacts how many copies are measured by dPCR. As an example, identical samples can have a two-fold difference in dPCR concentrations depending on denaturation state (one intact double-strand DNA molecule in one compartment is measured as one copy, two single-strand DNA molecules in two compartments is measured as two copies). The methods of the invention may include the use of two positive control reactions during the pre-amplification. The first positive control comprises positive control DNA and the assay, so that pre-amplification occurs. The second positive control comprises an equal amount of positive control DNA, without the primers. The first positive control and the second positive control are technical replicates with the exception that first positive control receives the primer(s).

Subsequently, the concentration of the replicate with primers is divided by the replicate with no primers to estimate the efficiency of assay. The efficiency calculated can be applied to the measurements performed on the actual samples. In effect, the invention provides a method to quantify the amount of target nucleic acid in the sample.

### Example

An exemplary method of the invention was used to simultaneously detect the ESR1 variants (D538G, Y537S, Y537C, Y537N, Y537H, Y537D, L536H, L536P, and L536R) described in FIGS. 1-10. Two ESR1 target sequences were amplified to produce a first and a second amplicon using a primer pair for each target sequence. The first amplicon included the nucleotides of the wildtype ESR1 nucleotide sequence encoding amino acids 529-552 of the ESR1 protein sequence. The second amplicon included the nucleotides of the wildtype ESR1 nucleotide sequence encoding amino acids 372-395 of the ESR1 protein sequence. Thus, between the two amplicons, the genomic location of each variant was included.

FIGS. 1-2 show the probe sequences, the ESR1 variants they interrogate, and the associated optical labels for each probe. FIG. 12 shows probe reaction concentrations for example assay designs. Targets are detected in channels with target specific probes at higher concentrations (e.g., 250 nM) and discrimination is facilitated with target specific probes at lower concentrations (e.g., 50 nM).

In the assay, the most common mutation, p.D538G, was detected based on the corresponding probe's optical label using a dedicated channel (FAM/Green). The next three most common mutations (p.Y537S, p.E380Q, and p.Y537N) were detected based on the fluorophore for that tranche of probes/variants using CY5/Crimson channel. For variant p.Y537C, a second probe was used with a different optical label, in this case, FAM detected by the Green channel. As shown in FIG. 1, the concentration of this second probe for p.Y537C was provided in a low concentration relative to the other probes. Continuing, in the ROX/Red channel, a tranche of three (3) less frequent mutations (p.Y537N, p.Y537H, and p.Y537D) were detected and in the fourth tranche three (3) less frequent mutations were detected in the ATTO550/Orange channel (p.L536H, p.L536P, and p.L536R).

As described above, all variant sequences were detected. For each variant, a characteristic dPCR signal was discerned relative to the expected wildtype sequence dPCR signal.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for detecting variant nucleic acids, the method comprising:
partitioning a sample comprising target nucleic acids into a plurality of partitions;
amplifying the nucleic acids in the partitions in the presence of a set of variant-specific probes each of which anneal to a respective variant, wherein each variant-specific probe includes a detectable label and wherein the set of probes has a number of distinct detectable labels that is lower than a number of the respective variants;
detecting signals from the partitions;
generating a plot of points representing the signals; and
identifying the presence of each variant in the sample from the presence of a corresponding cluster of points in the plot.

2. The method of claim 1, wherein the generating step comprises mapping the detected signals onto a space defined by the number of distinct detectable labels.

3. The method of claim 1, further comprising assigning a vector to each cluster of points, wherein each vector uniquely and specifically identifies one of the variants in the sample.

4. The method of claim 1, wherein at least one of the probes detects a number of the variants in the sample, and at least a second of the probes is specific to fewer than the number of the variants, is present at a different concentration than the one probe, and is used to discriminate among the variants by causing the points in the plot to form distinct clusters.

5. The method of claim 1, wherein two or more of the different variant sequences occur at positions on the target nucleic acid that will be amplified by one primer pair the amplifying step.

6. The method of claim 1, wherein the detectable label is an optical label.

7. The method of claim 1, wherein the presence of one or more variant sequences indicates a diseased state, optionally wherein the diseased state is cancer.

8. The method of claim 7, wherein the presence of one or more variant sequences indicates a minimal residual disease in the subject.

9. The method of claim 1, further comprising obtaining an estimate of relative abundances of the variants prior to the partitioning step and designing the variant-specific probes based on the estimate.

10. The method of claim 8, wherein identifying the presence or absence of one or more of the variant sequences indicates a progression or regression of the diseased state.

11. The method of claim 7, wherein the variants include tumor mutations determined by sequencing tumor nucleic acid from a tumor sample.

12. The method of claim 6, wherein the step of performing digital PCR further comprises using probes specific for a wild-type sequence.

13. The method of claim 1, further comprising assigning the variants to tranches and, for at least one tranche, providing at least one probe that detects multiple variants in the tranche and at least one probe that discriminates between the multiple variants in the tranche.

14. The method of claim 13, wherein the tranches are determined based on genomic position or information about probable relative frequency of the variants in the sample.

15. The method of claim 13, wherein each tranche is defined as a set of variants that can be amplified by one primer pair.

16. The method of claim 1, wherein the optical labels are selected from FAM, HEX, SUN, VIC, TAMRA, ATTO550, Cy5, ROX, ATTO700, Cy5.5, Yakima Yellow, ABY, JUN.

17. The method of claim 1, wherein the sample comprises cell-free DNA (cfDNA).

18. The method of claim 1, further comprising, prior to the partitioning step, identifying a first pair of first and second variants among the variants that form overlapping clusters on a 2D dPCR plot and designing a probe set that includes:
detection probes for both variants of the pair, wherein the detection probes both have a detection optical label of a first color; and
at least one discrimination probe having a discrimination optical label of a second colors.

19. The method of claim 18, wherein the amplifying step is performed with the detection probes and the discrimination probes present at different concentrations.

20. The method of claim 18, wherein the detection probes further include a third probe specific for a third variant, not of the pair, the third probe having an optical label of the first color.

21. The method of claim 20, wherein the plot comprises first, second, and third clusters from the respective first, second, and third variants, and wherein vectors from the origin of the plot through centroids of the clusters are non-orthogonal.

22. The method of claim 20, wherein the first, second, and third variants are located at positions on the target nucleic acid so as to all be amplified by one primer pair during the amplifying step.

23. The method of claim 1, wherein the probes include:
a first probe specific for a first variant and having a first optical label;
a second probe specific for a second variant and having the first optical label;
a third probe specific for a third variant and having the first optical label;
a WT probe specific for a wildtype sequence and having a second optical label; and
a discrimination probe specific for the first variant and having a third optical label.

24. The method of claim 23, further comprising generating a first two-color plot from the first and second optical labels, and identifying the presence of at least the first and second variants from first two-color plot.

25. The method of claim 24, further comprising generating a second two-color plot from the first and third optical labels, and discriminating the first variant sequence from at least the second variant based on a deviation of the second two-color plot from an expected dPCR two-color plot.
